(19) United States
(12) Patent Application Publication
(10) Pub. No.: US 2023/0172736 A1
(43) Pub. Date: Jun. 8, 2023

(54) TUBULAR MEDICAL INSTRUMENT TRANSFER DEVICE AND METHOD FOR MANUFACTURING TUBULAR MEDICAL INSTRUMENT TRANSFER DEVICE

(71) Applicant: KANEKA CORPORATION, Osaka (JP)

(72) Inventors: Kazuhiro SATO, Tokyo (JP); Shizuo ICHIMURA, Settsu-shi (JP)

(73) Assignee: KANEKA CORPORATION, Osaka (JP)

(21) Appl. No.: 18/103,688

(22) Filed: Jan. 31, 2023

Related U.S. Application Data
(63) Continuation of application No. PCT/JP2021/020595, filed on May 31, 2021.

(30) Foreign Application Priority Data
Aug. 3, 2020 (JP) 2020-131676

(51) Int. Cl. A61F 2/95; A61M 25/00

(52) U.S. Cl. CPC A61F 2/95; A61M 25/0009; A61F 2210/0014; C22F 1/006

(57) ABSTRACT

A method for manufacturing a tubular medical instrument transfer device which includes a tubular medical instrument and a tubular tube body comprise a step S1 for accommodating at least a part of the tubular medical instrument into a lumen of the tubular tube body and a step S2 for cooling the tubular medical instrument to a temperature of a martensitic phase transformation start temperature of the shape memory alloy+7° C. or less and a tubular medical instrument transfer device characterized in that a sliding load under 50° C. warm water and a sliding load under 25° C. warm water satisfy a relationship represented by Expression (1).

increase rate of sliding load [%]=(sliding load under 50° C. warm water [N]−sliding load under 25° C. warm water [N])/sliding load under 25° C. warm water [N]×100≤30[%] (1)

### TECHNICAL FIELD

The present invention relates to a tubular medical instrument transfer device that transfers a tubular medical instrument into a body, and a method for manufacturing the tubular medical instrument transfer device.

### BACKGROUND ART

In recent years, a treatment using a tubular medical instrument transfer device has been used as one of treatment methods for various diseases caused by stenosis or occlusion of a lumen in a living body such as a digestive tract such as a bile duct or a pancreatic duct, or a blood vessel such as an iliac artery. For example, a small hole is opened in the wrist, elbow, thigh, or the like, and the tubular medical instrument transfer device is inserted into the artery and brought to a lesion through the artery. The lesion is treated by expanding the tubular medical instrument contained in a tubular tube body at the lesion. This method is minimally invasive and imposes a small burden on patients, and thus, is one of treatment methods actively used in medical settings.

Meanwhile, the conventional tubular medical instrument transfer device is likely to have a phenomenon in which a tubular medical instrument that is relatively rigid sinks into a relatively soft tubular tube body after sterilization treatment or during a storage period. In a case where the tubular medical instrument is deployed in a state of sinking into the tubular tube body, a frictional force generated between the tubular medical instrument and the tubular tube body increases during deployment of the tubular medical instrument. For this reason, there is a problem that the tubular medical instrument transfer device itself may be damaged or the tubular medical instrument may be poorly deployed.

As a device capable of preventing the above-described sinking of the tubular medical instrument into the tubular tube body, a self-expandable stent feeding device provided with a reinforcement layer between an outer layer and an inner layer of an outer sheath is known (Patent Document 1).

The self-expandable stent feeding device described in Patent Document 1 can prevent a self-expandable stent from sinking into the outer sheath by providing the reinforcement layer between the outer layer and the inner layer of the outer sheath. For this reason, the diameter of the outer sheath on which the reinforcement layer is provided increases, and thus, it is difficult to reduce the diameter of the self-expandable stent feeding device. The reduction in the diameter of the transfer device is important for minimally invasive treatment, and thus, it has been desired to develop a transfer device that can prevent the self-expandable stent from sinking into the outer sheath and reduce a sliding load during deployment without using such a reinforcement layer.

### RELATED ART DOCUMENTS

### Patent Documents

Patent Document 1: JP-A-Hei-11-313893

### SUMMARY OF THE INVENTION

### Technical Problem

The present invention has been made in view of the above circumstances, and an object of the present invention is to provide a novel tubular medical instrument transfer device that can prevent a tubular medical instrument from sinking into a tubular tube body and decrease the sliding load during deployment of the tubular medical instrument, and a method for manufacturing the tubular medical instrument transfer device.

### Solutions to the Problems

The gist of one embodiment of a method for manufacturing a tubular medical instrument transfer device according to the present invention that can overcome the above problems is as follows. The method for manufacturing a tubular medical instrument transfer device according to the present invention is a method for manufacturing a tubular medical instrument transfer device including a tubular medical instrument that is made of a material containing a shape memory alloy, and a tubular tube body that is made of a material containing a thermoplastic resin, the method comprising: a step S1 for accommodating at least a part of the tubular medical instrument into a lumen of the tubular tube body; and a step S2 for cooling the tubular medical instrument to a temperature of a martensitic phase transformation start temperature of the shape memory alloy+7° C. or less. It is considered that at least a part of the shape memory alloy can undergo martensitic phase transformation by cooling the tubular medical instrument at a temperature of the martensitic phase transformation start temperature of the shape memory alloy+7° C. or less. With this configuration, the tubular medical instrument can be easily deformed even with low stress, whereby it is possible to reduce the sinking of the tubular medical instrument into the tubular tube body. Thus, it is possible to decrease the sliding load generated between the tubular medical instrument and the tubular tube body during deployment of the tubular medical instrument.

In step S2 of the method for manufacturing a tubular medical instrument transfer device, the tubular tube body is preferably cooled to a glass transition temperature of the thermoplastic resin or less.

The method for manufacturing a tubular medical instrument transfer device preferably includes step S3 for sterilizing the tubular medical instrument and the tubular tube body by heat, step S3 being performed after step S1 for accommodating at least a part of the tubular medical instrument into the lumen of the tubular tube body and before step S2 for cooling the tubular medical instrument to a temperature of the martensitic phase transformation start temperature of the shape memory alloy+7° C. or less.

It is preferable that at least a part of the tubular medical instrument is accommodated in the tubular tube body in contact with an inner wall of the tubular tube body.

It is preferable that the shape memory alloy is a nickel-titanium alloy.

It is preferable that the tubular medical instrument is a self-expandable stent.

The gist of one embodiment of a tubular medical instrument transfer device according to the present invention that can overcome the above problems is as follows. The tubular medical instrument transfer device according to the present invention includes: a tubular medical instrument made of a material containing a shape memory alloy; and a tubular tube body made of a material containing a thermoplastic resin, the tubular medical instrument being accommodated in a lumen of the tubular tube body, wherein the tubular medical instrument transfer device satisfies a relationship represented by following Expression (1) regarding a sliding load between the tubular medical instrument and the tubular tube body measured under 50° C. warm water (hereinafter referred to as “sliding load under 50° C. warm water”), and a sliding load between the tubular medical instrument and the tubular tube body measured under 25° C. warm water (hereinafter referred to as “sliding load under 25° C. warm water”). As a result, even when the tubular medical instrument transfer device is used in a body having a temperature higher than room temperature, a sliding load generated between the tubular medical instrument and the tubular tube body during deployment of the tubular medical instrument can be decreased.

increase rate of sliding load [%]=(sliding load under 50° C. warm water [N]−sliding load under 25° C. warm water [N])/sliding load under 25° C. warm water [N]×100≤30[%] (1)

It is preferable that the increase rate of the sliding load is greater than 0[%].

### Advantageous Effects of the Invention

The tubular medical instrument transfer device according to the present invention and the tubular medical instrument transfer device manufactured by the method for manufacturing a tubular medical instrument transfer device according to the present invention can prevent the tubular medical instrument from sinking into the tubular tube body and decreasing the sliding load generated between the tubular medical instrument and the tubular tube body during deployment of the tubular medical instrument.

### BRIEF DESCRIPTION OF THE DRAWINGS

FIG. 1 is a partial cross-sectional view illustrating an example of a tubular medical instrument transfer device according to an embodiment of the present invention.

FIG. 2 is a partial cross-sectional view illustrating an example of a tubular medical instrument transfer device according to an embodiment of the present invention.

FIG. 3 is a partial cross-sectional view illustrating a method for measuring the sliding load.

FIG. 4 illustrates the sliding loads [N] under 37° C. warm water in Comparative Examples 4 to 7, Comparative Examples 8 to 11, Comparative Examples 12 to 15, Examples 4 to 7, Examples 8 to 11, and Examples 12 to 15 by a bar graph.

### DESCRIPTION OF EMBODIMENTS

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

First, a method for manufacturing a tubular medical instrument transfer device according to the present invention will be described. The method for manufacturing a tubular medical instrument transfer device according to the present invention is a method for manufacturing a tubular medical instrument transfer device including a tubular medical instrument that is made of a material containing a shape memory alloy, and a tubular tube body that is made of a material containing a thermoplastic resin, the method comprising: a step S1 for accommodating at least a part of the tubular medical instrument into a lumen of the tubular tube body; and a step S2 for cooling the tubular medical instrument to a temperature of a martensitic phase transformation start temperature of the shape memory alloy+7° C. or less.

The tubular medical instrument is made of a material containing a shape memory alloy. The shape memory alloy refers to an alloy having property (hereinafter referred to as “radial force” in some cases) of, when heated to a certain temperature or more after being deformed, returning to its original shape before the deformation. A part of the tubular medical instrument can be made of a shape memory alloy, or the entire tubular medical instrument can be made of a shape memory alloy.

The tubular medical instrument is a tubular body preferably having a cylindrical shape.

The size of the tubular medical instrument may be appropriately set according to the inner diameter and the length of the blood vessel in a lesion.

The type of the tubular medical instrument is not particularly limited, and examples thereof include a stent, a stent graft, a prosthetic valve, and a balloon. A stent can be preferably used as the tubular medical instrument.

The shape of the stent is not particularly limited, and examples thereof include a coiled stent containing one liner member made of a material containing memory alloy, a stent obtained by processing a tube made of a material containing a shape memory alloy by laser cutting, a stent assembled by welding a linear member made of a material containing a shape memory alloy with a laser, and a stent formed by weaving a plurality of linear members made of a material containing a shape memory alloy.

As the shape memory alloy, a copper-aluminum-nickel alloy, a copper-zinc-aluminum alloy, or the like can be used, but preferably, the shape memory alloy contains a nickel-titanium alloy, and more preferably, the shape memory alloy is a nickel-titanium alloy. Using a nickel-titanium alloy among shape memory alloys can enhance strength, fatigue resistance, and corrosion resistance. When the tubular medical instrument is formed, one type of shape memory alloy may be selected and used from the shape memory alloys described above, or a plurality of types of shape memory alloys may be selected and used. For example, the tubular medical instrument can be formed of a material obtained by mixing a plurality of types of shape memory alloys. In addition, a part of the tubular medical instrument may be formed of one shape memory alloy, and the remaining part of the tubular medical instrument may be formed of another shape memory alloy.

The tubular tube body is made of a material containing a thermoplastic resin. The thermoplastic resin refers to a resin that has a property of softening and exhibiting plasticity when heated to a certain temperature or more and solidifying (glass transition temperature) when cooled to a certain temperature or less. Examples thereof include polyethylene, polypropylene, polystyrene, vinyl chloride resin, methyl methacrylate resin, nylon, polyamide, semi-aromatic polyamide, fluororesin, polycarbonate, and polyester resin. In order to reduce the sliding load generated between the tubular medical instrument and the tubular tube body, the tubular tube body preferably contains an olefin resin or a fluororesin, and preferably contains polytetrafluoroethylene (PTFE) which is known to have a low friction coefficient. When the tubular tube body is formed, one type of resin may be selected and used from the thermoplastic resins described above, or a plurality of types of thermoplastic resins may be selected and used. For example, the tubular tube body can be formed of a material obtained by mixing a plurality of types of thermoplastic resins, or can be formed of an alloy obtained by mixing a plurality of types of thermoplastic resins. In addition, a part of the tubular tube body may be formed of one thermoplastic resin, and the remaining part of the tubular tube body may be formed of another thermoplastic resin. Note that the tubular tube body may be formed of a material obtained by mixing a synthetic resin other than a thermoplastic resin and a thermoplastic resin.

The tubular tube body is a tubular body preferably having a cylindrical shape. The tubular tube body may have a single layer or a plurality of layers. In a case where the tubular tube body has a plurality of layers, the layers may be made of different materials so that the layers vary in hardness. For example, from the viewpoint of improving the operability of the tubular medical instrument transfer device, the hardness of an outer layer of the tubular tube body may be lower than the hardness of an inner layer. In addition, from the viewpoint of increasing the durability of the tubular medical instrument transfer device, the hardness of the outer layer of the tubular tube body may be higher than the hardness of the inner layer. When the tubular tube body has a plurality of layers, it is preferable to use, for example, an alloy of nylon 12 and semi-aromatic polyamide as a material constituting the outer layer, and to use PTFE as a material constituting the inner layer. With the configuration described above, the sliding load generated between the tubular medical instrument and the tubular tube body can be reduced, and the durability of the tubular medical instrument transfer device can be enhanced. More specifically, the tubular tube body may be cylindrical shape and composed of two layers: a first layer facing the outside and a second layer facing the lumen of the tubular tube body. It is preferred that the first layer consists of an alloy of polyamide 12 (Diamid X1988) and semi-aromatic polyamide (Grilamid TR55) and the second layer consists of Teflon PTFE DISP 30.

The size of the tubular tube body may be appropriately set in consideration of the size of the tubular medical instrument, the size of the lesion, the size of the blood vessel through which the tubular medical instrument transfer device passes, and the like. The outer diameter of the tubular tube body can be, for example, 1.75 mm, 1.80 mm, 1.85 mm, or the like. The inner diameter of the tubular tube body can be, for example, 1.55 mm, 1.60 mm, 1.65 mm, or the like. The length of the tubular tube body in the longitudinal direction of the tubular tube body can be 140 mm, 150 mm, 160 mm, or the like. The thickness of the tubular tube body can be 90 μm, 100 μm, 110 μm, or the like. When the tubular tube body is composed of two layers, an outer layer and an inner layer, the thickness of the outer layer can be 80 μm, 85 μm, 90 μm, or the like, and the thickness of the inner layer can be 10 μm, 15 μm, 20 μm, or the like.

The tubular tube body may be molded by a known method, and a method such as extrusion molding can be used, for example.

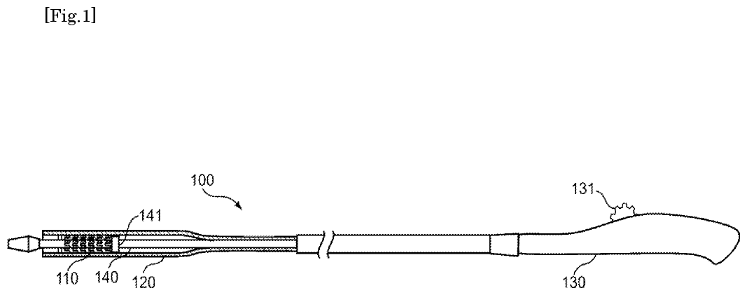

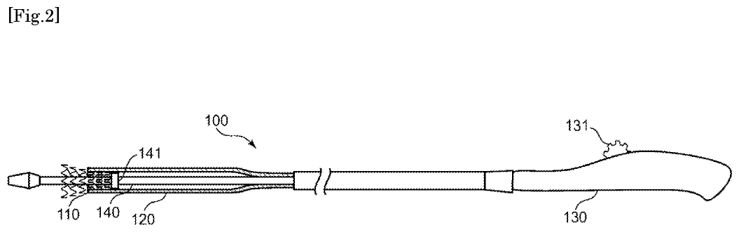

FIGS. 1 and 2 are partial cross-sectional views illustrating an example of a tubular medical instrument transfer device according to an embodiment of the present invention. FIG. 1 illustrates a state in which a tubular medical instrument is accommodated in a tubular tube body. FIG. 2 illustrates a state in which the tubular medical instrument is pushed out from the lumen of the tubular tube body.

As illustrated in FIG. 1, a tubular medical instrument transfer device 100 according to the embodiment of the present invention includes a tubular medical instrument 110 and a tubular tube body 120. The tubular tube body 120 has a proximal portion that is the operator's hand side and a distal portion that is a side opposite to the operator's hand, that is, a patient side. A half of the operator's hand side is defined as the proximal portion, and a half of the side opposite to the operator's hand is defined as the distal portion.

It is preferable that a proximal end of the tubular tube body 120 has an operation portion 130 to be operated by a user, and the operation portion 130 preferably has a shape easily gripped by the user during operation.

The tubular medical instrument transfer device 100 preferably includes an internal shaft 140 extending in the lumen of the tubular tube body 120, and the internal shaft 140 preferably includes a pusher member 141 that pushes out the tubular medical instrument 110. For example, the pusher member 141 can be disposed proximal to the tubular medical instrument 110. The pusher member 141 may have a hollow cylindrical shape, and the outer diameter of the pusher member 141 may be smaller than the inner diameter of the tubular tube body 120, and the outer diameter of the pusher member 141 may be equal to or greater than the inner diameter of the tubular medical instrument 110 accommodated in the tubular tube body 120. One end of the internal shaft 140 is exposed from the distal end portion of the tubular tube body 120, whereby a guide wire placed in the lumen of the internal shaft 140 can be advanced ahead of the tubular medical instrument transfer device 100. In addition, the other end of the internal shaft 140 may be attached to the operation portion 130, and a port through which the guide wire is inserted may be provided therein, for example.

As illustrated in FIG. 2, the tubular medical instrument transfer device 100 includes the operation portion 130 and the pusher member 141, and can push out the tubular medical instrument 110 from the tubular tube body 120 when the operation portion 130 is operated by the user. In this case, the user operates, for example, a thumbwheel 131 attached to the operation portion 130 to move the tubular tube body 120 to the proximal side. At this time, the tubular medical instrument 110 abuts the pusher member 141, by which only the tubular tube body 120 moves to the proximal side. Thus, the tubular medical instrument 110 can be deployed from the distal end of the tubular tube body 120 and placed on the lesion.

The operation portion 130 may be provided with the thumbwheel 131, a button, a lever, or the like for adjusting the positions of the internal shaft 140 and the pusher member 141 in the tubular tube body 120.

The configuration in which the tubular medical instrument 110 is placed on the lesion using the operation portion 130 and the pusher member 141 provided to the tubular medical instrument transfer device 100 has been described above. However, the configuration for placing the tubular medical instrument 110 on the lesion from the tubular tube body 120 is not limited thereto, and any known methods can be used.

The method for manufacturing the tubular medical instrument transfer device includes step S1 for accommodating at least a part of the tubular medical instrument into the lumen of the tubular tube body. In step S1, it is only sufficient that at least a part of the tubular medical instrument is accommodated in the lumen of the tubular tube body. The entire tubular medical instrument may be accommodated in the lumen of the tubular tube body.

The tubular medical instrument is preferably accommodated in the distal portion of the tubular tube body. The tubular medical instrument transfer device reaches the lesion through the blood vessel of the patient. Thereafter, the tubular medical instrument in the lumen of the tubular tube body is discharged from the tubular tube body and placed on the lesion by a user's operation on the operation portion. With the above configuration, the movement distance of the tubular medical instrument can be shortened, and the time in which the sliding load occurs between the tubular tube body and the tubular medical instrument can be decreased. Therefore, the user of the tubular medical instrument transfer device can easily place the tubular medical instrument at the lesion, and damage of the tubular medical instrument due to friction and poor deployment of the tubular medical instrument can be prevented.

The method for manufacturing the tubular medical instrument transfer device includes step S2 for cooling the tubular medical instrument to a temperature of a martensitic phase transformation start temperature of the shape memory alloy+7° C. or less. The martensitic phase refers to a crystal structure that appears in metal at a low temperature. Its crystal structure is weak against external force and relatively easy to deform, but it also has the property of returning to its original shape when the external force is removed. On the other hand, a crystal structure appearing at a high temperature is referred to as an austenite phase. The austenite phase has relatively high strength and exhibits a superelastic effect. The martensitic phase transformation start temperature generally refers to a temperature at which the martensitic phase appearing at a low temperature starts to appear, and it is considered that the martensitic phase partially starts to appear even at a temperature equal to a martensitic phase transformation start temperature of the shape memory alloy+7° C.

Step S2 for cooling the tubular medical instrument to a temperature of the martensitic phase transformation start temperature of the shape memory alloy+7° C. or less can be performed by putting the tubular medical instrument in a chamber set to a temperature of the martensitic phase transformation start temperature+7° C. or less or liquid nitrogen. In step S2, it is sufficient that the tubular medical instrument is cooled until the tubular medical instrument reaches a temperature of the martensitic phase transformation start temperature of the shape memory alloy+7° C. or less. The tubular medical instrument is preferably placed in a chamber set at a temperature of the martensitic phase transformation start temperature of the shape memory alloy+7° C. or less or liquid nitrogen for one minute or more, more preferably three minutes or more, and still more preferably five minutes or more. The upper limit of the time for placing the tubular medical instrument in a chamber set at a temperature of the martensitic phase transformation start temperature+7° C. or less or liquid nitrogen can be set to, for example, 24 hours or less, 12 hours or less, 8 hours or less, 4 hours or less, or 3 hours or less.

In step S2 for cooling the tubular medical instrument, the tubular medical instrument is cooled to a temperature of the martensitic phase transformation start temperature of the shape memory alloy included in the tubular medical instrument+7° C. or less. The cooling temperature in step S2 is more preferably of the martensitic phase transformation start temperature of the shape memory alloy included in the tubular medical instrument+5° C. or less, and still more preferably of the martensitic phase transformation start temperature of the shape memory alloy included in the tubular medical instrument+3° C. or less. The cooling temperature in step S2 may be of the martensitic phase transformation start temperature of the shape memory alloy included in the tubular medical instrument or less.

In step S2, it is only sufficient that the tubular medical instrument is cooled to a temperature of the martensitic phase transformation start temperature of the shape memory alloy included in the tubular medical instrument+7° C. or less. However, in step S2, it is preferable that the tubular tube body is also cooled to a temperature of the martensitic phase transformation start temperature of the shape memory alloy included in the tubular medical instrument+7° C. or less. In this case, step S2 for cooling the tubular medical instrument and the tubular tube body to a temperature of the martensitic phase transformation start temperature of the shape memory alloy+7° C. or less can be performed by putting the tubular medical instrument and the tubular tube body in a chamber set to a temperature of the martensitic phase transformation start temperature+7° C. or less or liquid nitrogen. In step S2, it is sufficient that the tubular medical instrument and the tubular tube body are cooled until the tubular medical instrument and the tubular tube body reach a temperature of the martensitic phase transformation start temperature of the shape memory alloy+7° C. or less. The tubular medical instrument and the tubular tube body are preferably placed in a chamber set at a temperature of the martensitic phase transformation start temperature of the shape memory alloy+7° C. or less or liquid nitrogen for one minute or more, more preferably three minutes or more, and still more preferably five minutes or more. The upper limit of the time for placing the tubular medical instrument and the tubular tube body in a chamber set at a temperature of the martensitic phase transformation start temperature+7° C. or less or liquid nitrogen can be set to, for example, 24 hours or less, 12 hours or less, 8 hours or less, 4 hours or less, or 3 hours or less. The cooling temperature in step S2 is more preferably of the martensitic phase transformation start temperature of the shape memory alloy included in the tubular medical instrument+5° C. or less, and still more preferably of the martensitic phase transformation start temperature of the shape memory alloy included in the tubular medical instrument+3° C. or less. The cooling temperature in step S2 may be of the martensitic phase transformation start temperature of the shape memory alloy included in the tubular medical instrument or less.

The method for manufacturing the tubular medical instrument transfer device is for preventing the tubular medical instrument which is relatively rigid from sinking into the tubular tube body which is relatively soft after the manufacture so as to reduce the sliding load generated during deployment. Therefore, it is preferable that steps S1 and S2 are performed in this order.

It is considered that at least a part of the shape memory alloy can undergo martensitic phase transformation by cooling the tubular medical instrument at a temperature of the martensitic phase transformation start temperature of the shape memory alloy+7° C. or less as described above. With this configuration, the tubular medical instrument can be easily deformed even with low stress, whereby generation of radial force can be prevented. Thus, it is possible to reduce the sinking of the tubular medical instrument into the tubular tube body, whereby the sliding load generated between the tubular medical instrument and the tubular tube body during deployment of the tubular medical instrument can be decreased.

In step S2 of the method for manufacturing a tubular medical instrument transfer device, the tubular tube body is preferably cooled to a glass transition temperature of the thermoplastic resin or less. When the tubular tube body that accommodates the tubular medical instrument is cooled to a glass transition temperature of the thermoplastic resin or less, the thermoplastic resin is cured and decreases in elastic modulus. With this configuration, the tubular tube body is less likely to be deformed even when external force is applied the tubular tube body, whereby it is possible to reduce the sinking of the tubular medical instrument into the tubular tube body. Thus, it is possible to decrease the sliding load generated between the tubular medical instrument and the tubular tube body during deployment of the tubular medical instrument. When the tubular tube body is composed of multiple layers and the resins constituting each layer are different, it is preferable that the tubular tube body is preferably cooled to a glass transition temperature of the thermoplastic resin contained in the thickest layer or less. When the tubular tube body has more than one layer of the same thickness, the tubular tube body is preferably cooled to a glass transition temperature of the thermoplastic resin contained in the layer with the largest cross-sectional area in the direction perpendicular to the longitudinal direction of the tubular tube body.

The method for manufacturing a tubular medical instrument transfer device preferably includes a sterilization step. Sterilization refers to an action or operation for achieving a state of killing or removing all proliferating microorganisms. As a method of sterilization, any known method may be used. For example, the method of sterilization may be selected from gas sterilization, electron beam sterilization, heat sterilization such as high-pressure steam sterilization (autoclave sterilization) and dry heat sterilization, radiation sterilization, and the like. The sterilization step is preferably performed after step S1 for accommodating at least a part of the tubular medical instrument into the lumen of the tubular tube body and before step S2 for cooling the tubular medical instrument to a temperature of the martensitic phase transformation start temperature of the shape memory alloy+7° C. or less.

The method for manufacturing a tubular medical instrument transfer device preferably includes step S3 for sterilizing the tubular medical instrument and the tubular tube body by heat, step S3 being performed after step S1 for accommodating at least a part of the tubular medical instrument into the lumen of the tubular tube body and before step S2 for cooling the tubular medical instrument to a temperature of the martensitic phase transformation start temperature of the shape memory alloy+7° C. or less.

In step S3, the tubular medical instrument and the tubular tube body are sterilized by heat. More specifically, the tubular medical instrument and the tubular tube body can be sterilized by a method such as EOG sterilization or electron beam sterilization, and it is preferable that the sterilization step includes a heat sterilization step. Steps S1, S3, and S2 are preferably performed in this order. The tubular tube body is softened by being heated to the glass transition temperature or more, and the tubular medical instrument is heated to an austenitic phase transformation end temperature or more, whereby the superelastic effect is further increased. Therefore, sinking of the tubular medical instrument after heat sterilization becomes remarkable, which may increase a sliding load generated between the tubular medical instrument and the tubular tube body. However, it is considered that at least a part of the shape memory alloy contained in the tubular medical instrument can undergo martensitic phase transformation by cooling the tubular medical instrument and the tubular tube body to a temperature of the martensitic phase transformation start temperature of the shape memory alloy included in the tubular medical instrument+7° C. or less after heat sterilization. Accordingly, the tubular medical instrument can be easily deformed even with low stress, and thus, it is possible to suppress the generation of radial force. With this configuration, the sinking of the tubular medical instrument into the tubular tube body can be reduced, and thus, it is possible to decrease the sliding load generated between the tubular medical instrument and the tubular tube body during deployment of the tubular medical instrument. When the tubular tube body is cooled to the glass transition temperature or less, a synergistic effect is generated due to an increase in the hardness of the tubular tube body, whereby the sinking of the tubular medical instrument into the tubular tube body can be reduced, and the sliding load generated during deployment of the tubular medical instrument can be decreased.

The temperature for heat sterilization in step S3 may be any temperature that can kill bacteria and may be appropriately set. The lower limit of the temperature for heat sterilization can be set to, for example, 40° C. or more, 45° C. or more, or 50° C. or more. The upper limit of the temperature for heat sterilization can be set to, for example, 130° C. or less, 120° C. or less, or 110° C. or less.

It is preferable that at least a part of the tubular medical instrument is accommodated in the tubular tube body in contact with an inner wall of the tubular tube body. The configuration in which at least a part of the tubular medical instrument is accommodated in the tubular tube body in contact with an inner wall of the tubular tube body means a configuration in which another member is not disposed between the tubular medical instrument and the tubular tube body at that portion, and thus, the diameter at that portion is easily reduced.

The stents used as the tubular medical instrument can be generally classified into a balloon-expandable stent and a self-expandable stent based on the expandable mechanism. The balloon-expandable stent is configured so that the stent is delivered to the lesion with the stent attached to the outer surface of the balloon and the stent is expanded by the balloon at the lesion. The self-expandable stent is configured so that the stent is inserted in a tubular body having a sheath member that suppresses expansion of the stent and is delivered to a lesion site, and expands by itself when the sheath member is removed at the lesion site.

The method for manufacturing a tubular medical instrument transfer device can be optimally used when the tubular medical instrument is a self-expandable stent. The self-expandable stent expands immediately after the self-expandable stent is released from the tubular medical instrument transfer device. When the tubular medical instrument is a self-expandable stent, force by which the tubular medical instrument is to be deployed always acts on the tubular tube body, so that the tubular medical instrument is likely to sink into the inner wall surface of the tubular tube body. On the other hand, it is considered that at least a part of the shape memory alloy included in the tubular medical instrument can start martensitic phase transformation due to the execution of the manufacturing method described above. It is considered that the force acting on the tubular tube body is suppressed by the martensitic phase transformation of at least a part of the tubular medical instrument. With this configuration, the sinking of the tubular medical instrument into the tubular tube body can be reduced, and thus, it is possible to decrease the sliding load generated between the tubular medical instrument and the tubular tube body during deployment of the tubular medical instrument.

The self-expandable stent can be manufactured, for example, by cutting a cylindrical pipe made of a nickel-titanium alloy with a laser, increasing the diameter of the pipe, heat treating the pipe to form a desired shape, and finally electropolishing the pipe.

The method for manufacturing a tubular medical instrument transfer device according to the embodiment of the present invention has been described so far. Next, a tubular medical instrument transfer device according to an embodiment of the present invention will be described.

The tubular medical instrument transfer device according to the present invention includes: a tubular medical instrument made of a material containing a shape memory alloy; and a tubular tube body made of a material containing a thermoplastic resin, the tubular medical instrument being accommodated in a lumen of the tubular tube body, wherein the tubular medical instrument transfer device satisfies a relationship represented by following Expression (1) regarding a sliding load between the tubular medical instrument and the tubular tube body measured under 50° C. warm water (hereinafter referred to as “sliding load under 50° C. warm water”), and a sliding load between the tubular medical instrument and the tubular tube body measured under 25° C. warm water (hereinafter referred to as “sliding load under 25° C. warm water”). With this configuration, the sinking of the tubular medical instrument into the tubular tube body can be reduced, and thus, it is possible to decrease the sliding load generated between the tubular medical instrument and the tubular tube body during deployment of the tubular medical instrument.

increase rate of sliding load [%]=(sliding load under 50° C. warm water [N]−sliding load under 25° C. warm water [N])/sliding load under 25° C. warm water [N]×100≤30[%] (1)

In the following, results of actually measuring a sliding load generated between a tubular medical instrument and a tubular tube body in a tubular medical instrument transfer device manufactured according to the embodiment of the present invention (Examples 1 to 3) and results of actually measuring a sliding load generated between a tubular medical instrument and a tubular tube body in a tubular medical instrument transfer device manufactured according to the conventional method (Comparative Examples 1 to 3) will be described.

Tubular medical instrument transfer devices according to Examples 1 to 3 in the following Table 1 are manufactured by the method described as the method for manufacturing a tubular medical instrument transfer device. More specifically, the tubular medical instrument transfer devices are manufactured by performing step S1 for accommodating the entire of the tubular medical instrument into a lumen of the tubular tube body, step S3 for sterilizing the tubular medical instrument and the tubular tube body by heat, and step S2 for cooling the tubular medical instrument and the tubular tube body to a temperature equal to a martensitic phase transformation start temperature of the shape memory alloy+3° C. in this order, and after step S2 for cooling, the tubular medical instrument transfer devices are stored at normal temperature (25° C.). The temperature equal to the martensitic phase transformation start temperature of the shape memory alloy+3° C. is a temperature of the glass transition temperature of the thermoplastic resin contained in the tubular tube body−67° C.

The tubular medical instrument transfer devices according to Comparative Examples 1 to 3 in Table 1 are manufactured by performing step S1 for accommodating the entire of the tubular medical instrument into a lumen of the tubular tube body and step S3 for sterilizing the tubular medical instrument and the tubular tube body by heat in this order. In Comparative Examples, step S2 for cooling the tubular medical instrument and the tubular tube body is not performed, and after step S3 for heat sterilization, the tubular medical instrument transfer devices are stored at normal temperature (25° C.).

Note that the tubular medical instrument used for manufacturing the tubular medical instrument transfer device of each of Examples 1 to 3 and Comparative Examples 1 to 3 is a self-expandable stent obtained by cutting out a tube made of a material containing a nickel-titanium alloy as a shape memory alloy with a laser and processing the cut tube. The diameter of the tubular medical instrument before being accommodated in the lumen of the tubular tube body is 10 mm, and the length of the tubular medical instrument is 100 mm. The tubular tube body has an outer layer made of nylon 12 and an inner layer made of PTFE. The tubular tube body has an inner diameter of 1.61 mm and an outer diameter of 1.81 mm. The thickness of the inner layer formed of PTFE is 15 μm. In addition, the shape of the self-expandable stent which is a tubular medical instrument is the same between Examples and Comparative Examples. The martensitic phase transformation start temperature of the shape memory alloy included in the tubular medical instrument of each of Examples 1 to 3 and Comparative Examples 1 to 3 is −35° C. The glass transition temperature of the thermoplastic resin contained in the tubular tube body of each of Examples 1 to 3 and Comparative Examples 1 to 3 is 35° C. In step S3 for heat sterilization, EOG sterilization was performed at a temperature of 60° C. and a humidity of 60% for 30 hours.

Table 1 below shows results (hereinafter referred to as “sliding load under 25° C. warm water” in some cases) of measuring sliding loads [N] generated between the tubular medical instruments and the tubular tube bodies measured under 25° C. warm water in the tubular medical instrument transfer devices according to Examples 1 to 3 and the tubular medical instrument transfer devices according to Comparative Examples 1 to 3, and results (hereinafter referred to as “sliding load under 50° C. warm water” in some cases) of measuring sliding loads [N] generated between the tubular medical instruments and the tubular tube bodies measured under 50° C. warm water in the tubular medical instrument transfer devices according to Examples 1 to 3 and the tubular medical instrument transfer devices according to Comparative Examples 1 to 3. Table 1 also shows amounts of change [N] between the sliding load measured under 25° C. warm water and the sliding load measured under 50° C. warm water (sliding load [N] under 50° C. warm water−sliding load [N] under 25° C. warm water), and increase rates [%] of sliding load between the sliding load measured under 25° C. warm water and the sliding load measured under 50° C. warm water ((sliding load under 50° C. warm water [N]−sliding load under 25° C. warm water [N])/sliding load under 25° C. warm water [N]×100).

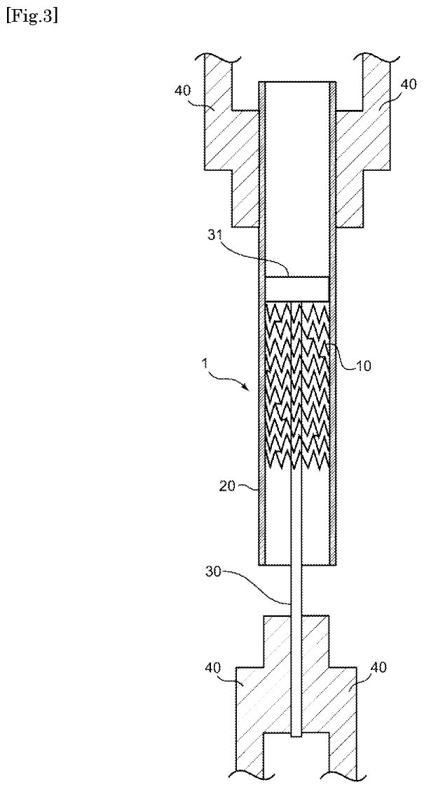

Next, a method for measuring the sliding load will be described with reference to FIG. 3. First, Sample 1, which is a tubular medical instrument 10 is accommodated in the lumen of a tubular tube body 20, is prepared. One end of the tubular tube body 20 is fixed to a tension load measuring device 40, and a support member 30 for supporting the tubular medical instrument 10 is placed in the lumen of the tubular tube body 20. A pusher member 31 which has hollow cylindrical shape is provided at one end of the support member 30, and the tubular medical instrument 10 placed in the lumen of the tubular tube body 20 is pushed out by the pusher member 31 provided to the support member 30. The other end of the support member 30 is exposed to the outside of the tubular tube body 20 and fixed to the tension load measuring device 40. With this state, an S-S curve when the tubular tube body 20 is pulled at a speed of 50 mm/min with the position of the support member 30 fixed is obtained by the tension load measuring device 40. In the present specification, a peak of the S-S curve is defined as a sliding load [N].

The sliding load under 25° C. warm water is a load measured in a state where the tubular medical instrument and the tubular tube body are immersed in warm water adjusted to 25° C. In measuring the sliding load under 25° C. warm water, it is preferable to measure the sliding load after immersing the Sample 1 in 25° C. warm water until the temperature of the Sample 1 reaches 25° C. For example, the sliding load should be measured after 30 seconds or more of immersion of the Sample 1 in warm water adjusted to 25° C.

The sliding load under 50° C. warm water is a load measured in a state where the tubular medical instrument and the tubular tube body are immersed in warm water adjusted to 50° C. In measuring the sliding load under 50° C. warm water, it is preferable to measure the sliding load after immersing the Sample 1 in 50° C. warm water until the temperature of the Sample 1 reaches 50° C. For example, the sliding load should be measured after 30 seconds or more of immersion of the Sample 1 in warm water adjusted to 50° C.

TABLE 1

| | sliding load | sliding load | | |
|---|---|---|---|---|
| | under 25° C. | under 50° C. | amounts | increase |
| | warm water | warm water | of change | rates |
| | [N] | [N] | [N] | [%] |
| Example 1 | 6.31 | 8.09 | 1.78 | 28.2 |
| Example 2 | 5.89 | 7.49 | 1.6 | 27.2 |
| Example 3 | 5.92 | 7.28 | 1.36 | 23.0 |
| Comparative | 6.67 | 9.75 | 3.08 | 46.2 |
| Example 1 | | | | |
| Comparative | 5.86 | 9.09 | 3.23 | 55.1 |
| Example 2 | | | | |
| Comparative | 5.92 | 7.97 | 2.05 | 34.6 |
| Example 3 | | | | |

As shown in Table 1, the sliding loads of the tubular medical instrument transfer devices of Examples 1 to 3 under 50° C. warm water are within 7.28 to 8.09 [N], whereas the sliding loads of the tubular medical instrument transfer devices of Comparative Examples 1 to 3 under 50° C. warm water are within 7.97 to 9.75 [N]. This result indicates that the tubular medical instrument transfer devices according to Examples 1 to 3 tend to decrease the sliding load between the tubular medical instrument and the tubular tube body as compared with the tubular medical instrument transfer devices according to Comparative Examples 1 to 3.

In addition, the minimum value of the increase rate of the sliding loads of the tubular medical instrument transfer devices of Examples 1 to 3 is 23.0[%], whereas the maximum value of the increase rate of the sliding loads of the tubular medical instrument transfer devices of Comparative Examples 1 to 3 is 55.1[%]. This result indicates that the tubular medical instrument transfer devices according to Examples 1 to 3 can suppress the increase rate of the sliding load by a maximum of 32.1[%] in comparison with the tubular medical instrument transfer devices according to Comparative Examples 1 to 3.

Furthermore, the increase rate of the sliding load of the tubular medical instrument transfer device is 28.2[%] in Example 1, 27.2[%] in Example 2, and 23.0[%] in Example 3, whereas it is 46.2[%] in Comparative Example 1, 55.1[%] in Comparative Example 2, and 34.6[%] in Comparative Example 3. As described above, the tubular medical instrument transfer device according to the embodiment of the present invention includes: a tubular medical instrument made of a material containing a shape memory alloy; and a tubular tube body made of a material containing a thermoplastic resin, the tubular medical instrument being accommodated in a lumen of the tubular tube body, the tubular medical instrument transfer device satisfying a relationship represented by following Expression (1) regarding a sliding load under 50° C. warm water and a sliding load under 25° C. warm water. With this configuration, it is possible to decrease the sliding load generated between the tubular medical instrument and the tubular tube body during deployment of the tubular medical instrument.

increase rate of sliding load [%]=(sliding load under 50° C. warm water [N]−sliding load under 25° C. warm water [N])/sliding load under 25° C. warm water [N]×100≤30[%] (1)

The increase rate of the sliding load can be greater than 0[%], and may be, for example, 5[%] or more or 10[%] or more. The smaller the increase rate [%] of the sliding load is, the more preferable it is.

In the following, Comparative Examples 4 to 7, Comparative Examples 8 to 15, and Examples 4 to 15 will be described. Comparative examples 4 to 7 each indicate a result of measuring a sliding load generated between a tubular medical instrument and a tubular tube body of a tubular medical instrument transfer device not subjected to the heat sterilization step S3 and the cooling step S2 under warm water at 37° C. which is closer to the body temperature. Comparative Examples 8 to 15 each indicate a result of measuring a sliding load generated between a tubular medical instrument and a tubular tube body of a tubular medical instrument transfer device manufactured by a conventional method under warm water at 37° C. which is closer to the body temperature. Examples 4 to 15 each indicate a result of measuring a sliding load generated between a tubular medical instrument and a tubular tube body of a tubular medical instrument transfer device according to the embodiment of the present invention under warm water at 37° C. which is closer to the body temperature.

The tubular medical instrument transfer devices according to Comparative Examples 4 to 7 in Table 2 are manufactured by performing only step S1 for accommodating the entire of the tubular medical instrument into a lumen of the tubular tube body. In Comparative Examples 4 to 7, neither step S2 for cooling the tubular medical instrument and the tubular tube body nor step S3 for heat sterilization is performed. They are stored at room temperature (25° C.) after the completion of step S1.

The tubular medical instrument transfer devices according to Comparative Examples 8 to 11 in Table 2 are manufactured by performing step S1 for accommodating the entire of the tubular medical instrument into a lumen of the tubular tube body and step S3 for sterilizing the tubular medical instrument and the tubular tube body by heat in this order. In Comparative Examples 8 to 11, step S2 for cooling the tubular medical instrument and the tubular tube body is not performed, and after step S3 for heat sterilization, the tubular medical instrument transfer devices are stored at normal temperature (25° C.).

The tubular medical instrument transfer devices according to Comparative Examples 12 to 15 in Table 2 are manufactured by performing step S1 for accommodating the entire of the tubular medical instrument into a lumen of the tubular tube body, step S3 for sterilizing the tubular medical instrument and the tubular tube body by heat, and step S2 for cooling the tubular medical instrument and the tubular tube body to a temperature equal to a martensitic phase transformation start temperature of the shape memory alloy+39° C. in this order, and after step S2 for cooling, the tubular medical instrument transfer devices are stored at normal temperature (25° C.). The temperature equal to the martensitic phase transformation start temperature of the shape memory alloy+39° C. is a temperature of the glass transition temperature of the thermoplastic resin contained in the tubular tube body−31° C.

The tubular medical instrument transfer devices according to Examples 4 to 7 in Table 2 are manufactured by performing step S1 for accommodating the entire of the tubular medical instrument into a lumen of the tubular tube body, step S3 for sterilizing the tubular medical instrument and the tubular tube body by heat, and step S2 for cooling the tubular medical instrument and the tubular tube body to a temperature equal to a martensitic phase transformation start temperature of the shape memory alloy+3° C. in this order, and after step S2 for cooling, the tubular medical instrument transfer devices are stored at normal temperature (25° C.). The temperature equal to the martensitic phase transformation start temperature of the shape memory alloy+3° C. is a temperature of the glass transition temperature of the thermoplastic resin contained in the tubular tube body−67° C.

The tubular medical instrument transfer devices according to Examples 8 to 11 in Table 2 are manufactured by performing step S1 for accommodating the entire of the tubular medical instrument into a lumen of the tubular tube body, step S3 for sterilizing the tubular medical instrument and the tubular tube body by heat, and step S2 for cooling the tubular medical instrument and the tubular tube body to a temperature equal to a martensitic phase transformation start temperature of the shape memory alloy−45° C. in this order, and after step S2 for cooling, the tubular medical instrument transfer devices are stored at normal temperature (25° C.). The temperature equal to the martensitic phase transformation start temperature of the shape memory alloy−45° C. is a temperature of the glass transition temperature of the thermoplastic resin contained in the tubular tube body−115° C.

The tubular medical instrument transfer devices according to Examples 12 to 15 in Table 2 are manufactured by performing step S1 for accommodating the entire of the tubular medical instrument into a lumen of the tubular tube body, step S3 for sterilizing the tubular medical instrument and the tubular tube body by heat, and step S2 for cooling the tubular medical instrument and the tubular tube body to a temperature equal to a martensitic phase transformation start temperature of the shape memory alloy−161° C. in this order, and after step S2 for cooling, the tubular medical instrument transfer devices are stored at normal temperature (25° C.). The temperature equal to the martensitic phase transformation start temperature of the shape memory alloy−161° C. is a temperature of the glass transition temperature of the thermoplastic resin contained in the tubular tube body−231° C.

Note that the tubular medical instrument used for manufacturing the tubular medical instrument transfer device of each of Comparative Examples 4 to 7, Comparative Examples 8 to 15, and Examples 4 to 15 is a self-expandable stent obtained by cutting out a tube made of a material containing a nickel-titanium alloy as a shape memory alloy with a laser and processing the cut tube. The diameter of the tubular medical instrument before being accommodated in the lumen of the tubular tube body is 10 mm, and the length of the tubular medical instrument is 100 mm. The tubular tube body has an outer layer made of nylon 12 and an inner layer made of PTFE. The tubular tube body has an inner diameter of 1.61 mm and an outer diameter of 1.81 mm. The thickness of the inner layer formed of PTFE is 15 μm. In addition, the shape of the self-expandable stent which is a tubular medical instrument is the same between Examples and Comparative Examples. The martensitic phase transformation start temperature of the shape memory alloy included in the tubular medical instrument is −35° C. The glass transition temperature of the thermoplastic resin contained in the tubular tube body is 35° C. In step S3 for heat sterilization, EOG sterilization was performed at a temperature of 60° C. and a humidity of 60% for 30 hours.

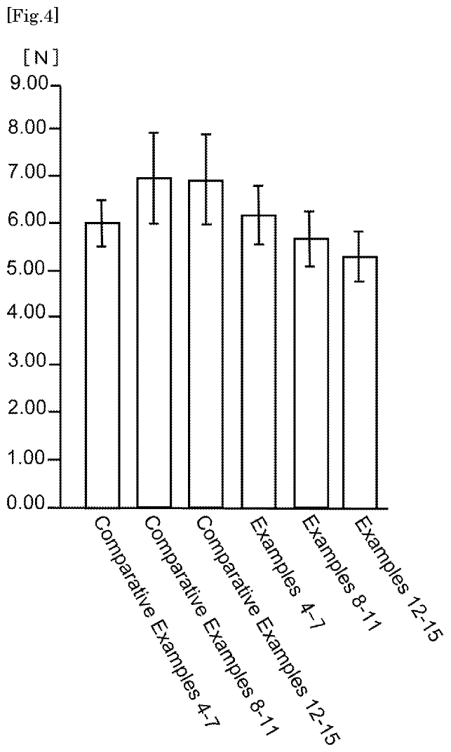

Table 2 shows the results of measuring the sliding loads [N] (hereinafter referred to as a “sliding load under 37° C. warm water” in some cases) generated between the tubular medical instruments and the tubular tube bodies of the tubular medical instrument transfer devices of Comparative Examples 4 to 7, Comparative Examples 8 to 15, and Examples 4 to 15 under 37° C. warm water, and averages of the sliding loads under 37° C. warm water in Comparative Examples 4 to 7, Comparative Examples 8 to 11, Comparative Examples 12 to 15, Examples 4 to 7, Examples 8 to 11, and Examples 12 to 15. FIG. 4 illustrates the sliding loads [N] under 37° C. warm water in Comparative Examples 4 to 7, Comparative Examples 8 to 11, Comparative Examples 12 to 15, Examples 4 to 7, Examples 8 to 11, and Examples 12 to 15 by a bar graph. The sliding load under 37° C. warm water is a load measured in a state where the tubular medical instrument and the tubular tube body are immersed in warm water adjusted to 37° C. In measuring the sliding load under 37° C. warm water, it is preferable to measure the sliding load after immersing the Sample 1 in 37° C. warm water until the temperature of the Sample 1 reaches 37° C. For example, the sliding load should be measured after 30 seconds or more of immersion of the Sample 1 in warm water adjusted to

TABLE 2

| | | | | Sliding load under 37° C. | |
|---|---|---|---|---|---|
| | | | | warm water [N] | |
| | | | | (martensitic phase | |
| | | | | transformation start | |
| Sliding load under 37° C. | | Sliding load under 37° C. | | temperature +39° C., | |
| warm water [N] | | warm water [N] | | glass transition | |
| (No heating, no cooling) | | (No cooling) | | temperature −31° C.) | |
| Comparative | 5.24 | Comparative | 6.04 | Comparative | 5.96 |
| Example 4 | | Example 8 | | Example 12 | |
| Comparative | 5.86 | Comparative | 6.66 | Comparative | 7.26 |
| Example 5 | | Example 9 | | Example 13 | |
| Comparative | 6.46 | Comparative | 8.30 | Comparative | 8.16 |
| Example 6 | | Example 10 | | Example 14 | |
| Comparative | 6.08 | Comparative | 6.88 | Comparative | 6.48 |
| Example 7 | | Example 11 | | Example 15 | |
| Average | 5.91 | Average | 6.97 | Average | 6.97 |
| Sliding load under 37° C. | | Sliding load under 37° C. | | Sliding load under 37° C. | |
| warm water [N] | | warm water [N] | | warm water [N] | |
| (martensitic phase | | (martensitic phase | | (martensitic phase | |
| transformation start | | transformation start | | transformation start | |
| temperature +3° C., | | temperature −45° C., | | temperature −161° C., | |
| glass transition | | glass transition | | glass transition | |
| temperature −67° C.) | | temperature −115° C.) | | temperature −231° C.) | |
| Example 4 | 5.52 | Example 8 | 5.54 | Example 12 | 4.80 |
| Example 5 | 6.22 | Example 9 | 5.70 | Example 13 | 5.72 |
| Example 6 | 7.12 | Example 10 | 6.54 | Example 14 | 5.86 |
| Example 7 | 6.12 | Example 11 | 5.12 | Example 15 | 5.16 |
| Average | 6.25 | Average | 5.73 | Average | 5.39 |

As shown in Comparative Examples 4 to 7 in Table 2 and FIG. 4, the average of the sliding loads of the tubular medical instrument transfer devices not subjected to step S3 for heat sterilization under 37° C. warm water is 5.91 N. As indicated in Comparative Examples 8 to 11, the average of the sliding loads of the tubular medical instrument transfer devices under 37° C. warm water increases to 6.97 N when cooling step S2 is not performed after heat sterilization step S3. In addition, as indicated in Comparative Examples 12 to 15, the average of the sliding loads under 37° C. warm water remains 6.97 N when only the step of cooling to the martensitic phase transformation start temperature of the shape memory alloy+39° C. and to a temperature of the glass transition temperature of the thermoplastic resin−31° C. is performed.

However, the average of the sliding loads under 37° C. warm water when the step of cooling to a temperature of a martensitic phase transformation start temperature+3° C. and to a temperature of the glass transition temperature−67° C. is performed is 6.25 N. This shows that the average decreases to around the average (5.91 N) of the sliding loads under 37° C. warm water in Comparative Examples 4 to 7 in which the heat sterilization step is not performed. As described above, when the cooling temperature in cooling step S2 is set to about the martensitic phase transformation start temperature+3° C., the sliding load under 37° C. warm water can be reduced to about the average value of the sliding loads under 37° C. warm water before heat sterilization.

In addition, the average of sliding loads under 37° C. warm water is 5.73 N in Examples 8 to 11 in which the step of cooling to the temperature of the martensitic phase transformation start temperature−45° C. and to the temperature of the glass transition temperature−115° C. is performed. In addition, the average of sliding loads under 37° C. warm water is 5.39 N in Examples 12 to 15 in which the step of cooling to the temperature of the martensitic phase transformation start temperature−161° C. and to the temperature of the glass transition temperature−231° C. is performed. As described above, when the cooling temperature in cooling step S2 is set to about the martensitic phase transformation start temperature−45° C., the sliding load under 37° C. warm water can be made lower than that before heat sterilization.

Furthermore, it can be seen from Examples 4 to 7, Examples 8 to 11, and Examples 12 to 15 that, the lower the cooling temperature in the cooling step is, the more the sliding load generated between the tubular tube body and the tubular medical instrument can be reduced. In particular, when the step of cooling to the temperature of the martensitic phase transformation start temperature of the shape memory alloy−161° C. and to the temperature of the glass transition temperature of the thermoplastic resin contained in the tubular tube body−231° C. is performed (Examples 12 to 15), most of the shape memory alloy can be transformed into the martensitic phase, so that the sliding load can be easily reduced.

As described above, it can be seen that the sliding load under 37° C. warm water between the tubular medical instrument and the tubular tube body which has been increased due to the execution of the heat sterilization step can be reduced by performing the step of cooling to a temperature of the martensitic phase transformation start temperature+7° C. or less after the execution of the heat sterilization step (Examples 4 to 7, Examples 8 to 11, Examples 12 to 15).

The temperature of 37° C. is a temperature close to the body temperature of the human body. It can be seen from the above results that the tubular medical instrument transfer device according to the embodiment of the present invention can have a sliding load lower than that of the tubular medical instrument transfer device (Comparative Examples 8 to 11) manufactured by the conventional method even after being inserted into a blood vessel of a human body.

Furthermore, the average of the sliding loads in Examples 8 to 11 in which the step of cooling was performed after heat sterilization and the average of the sliding loads in Examples 12 to 15 in which the step of cooling was performed after heat sterilization were lower than the average of the sliding loads in Comparative Examples 4 to 7 in which the heat sterilization step was not performed. From the above, it is considered that, when a sterilization method other than heat sterilization is used, the sliding load can also be reduced by performing cooling step S2 after step S1.

As described above, even when the tubular medical instrument transfer device according to the present invention is used in a body having a temperature higher than room temperature, a sliding load generated between the tubular medical instrument and the tubular tube body during deployment of the tubular medical instrument can be decreased.

As described above, the tubular medical instrument transfer device and the method for manufacturing the tubular medical instrument transfer device according to the present invention can prevent the tubular medical instrument from sinking into the tubular tube body and decrease the sliding load during deployment of the tubular medical instrument.

This application claims the benefit of the priority date of Japanese patent application No. 2020-131676 filed on Aug. 3, 2020. All of the contents of the Japanese patent application No. 2020-131676 filed on Aug. 3, 2020 are incorporated by reference herein.

### REFERENCE SIGNS LIST

1: Sample
10: Tubular medical instrument
20: Tubular tube body
30: Support member
31: Pusher member
40: Tension load measuring device
100: Tubular medical instrument transfer device
110: Tubular medical instrument
120: Tubular tube body
130: Operation portion
131: Thumbwheel
140: Internal shaft
141: Pusher member

1. A method for manufacturing a tubular medical instrument transfer device including a tubular medical instrument that is made of a material containing a shape memory alloy, and a tubular tube body that is made of a material containing a thermoplastic resin, the method comprising:
a step S1 for accommodating at least a part of the tubular medical instrument into a lumen of the tubular tube body; and
a step S2 for cooling the tubular medical instrument to a temperature of a martensitic phase transformation start temperature of the shape memory alloy+7° C. or less.

2. The method for manufacturing a tubular medical instrument transfer device according to claim 1, wherein, in the step S2, the tubular tube body is cooled to a glass transition temperature of the thermoplastic resin or less.

3. The method for manufacturing a tubular medical instrument transfer device according to claim 1, further comprising:
a step S3 for sterilizing the tubular medical instrument and the tubular tube body by heat, the step S3, wherein the step S3 is performed after the step S1 and before the step S2.

4. The method for manufacturing a tubular medical instrument transfer device according to claim 1, wherein the part of the tubular medical instrument is accommodated into the lumen of the tubular tube body, such that the part of the tubular medical instrument comes in contact with an inner wall of the tubular tube body.

5. The method for manufacturing a tubular medical instrument transfer device according to claim 1, wherein the shape memory alloy is a nickel-titanium alloy.

6. The method for manufacturing a tubular medical instrument transfer device according to claim 1, wherein the tubular medical instrument is a self-expandable stent.

7. A tubular medical instrument transfer device comprising:
a tubular medical instrument made of a material containing a shape memory alloy; and
a tubular tube body made of a material containing a thermoplastic resin, the tubular medical instrument being accommodated in a lumen of the tubular tube body,
wherein the tubular medical instrument transfer device satisfies a relationship represented by Expression (1):
increase rate of sliding load [%]=(sliding load under 50° C. warm water [N]−sliding load under 25° C. warm water [N])/sliding load under 25° C. warm water [N]×100≤30[%], (1)
where the “sliding load under 50° C. warm water” indicates a sliding load between the tubular medical instrument and the tubular tube body measured under 50° C. warm water, and the “sliding load under 25° C. warm water” indicates a sliding load between the tubular medical instrument and the tubular tube body measured under 25° C. warm water.

8. The tubular medical instrument transfer device according to claim 7, wherein the increase rate of the sliding load is greater than 0%.
